(19) United States
(12) Patent Application Publication
(10) Pub. No.: US 2026/0298933 A1
(43) Pub. Date: Oct. 1, 2026

(54) CERVICAL CANCER DETECTION PROTEINS AND METHODS OF USE THEREOF

(71) Applicant: Novelna Inc., Palo Alto, CA (US)

(72) Inventor: Ashkan Afshin, Santa Clara, CA (US)

(21) Appl. No.: 19/164,490

(22) PCT Filed: Mar. 12, 2024
(86) PCT No.: PCT/US2024/019550

§ 371 Date: Sep. 11, 2025

Related U.S. Application Data
(60) Provisional application No. 63/451,625, filed on Mar. 12, 2023.

(51) Int. Cl. G01N 33/5755

(52) U.S. Cl. CPC G01N 33/5755

(57) ABSTRACT

Methods of evaluating a subject for cervical cancer or detecting cervical cancer in a subject, the methods comprising determining in a biological sample from the subject a concentration of one or more proteins selected from Table 1. The methods may further comprise applying a classifier to the concentration of the one or more proteins that identifies whether the concentration of the one or more proteins is indicative of the subject having cervical cancer. In addition, methods of treatment comprising administering a treatment to the subject when the subject is evaluated or detected to have cervical cancer.

### CROSS-REFERENCE TO RELATED APPLICATIONS

This application claims benefit of U.S. Provisional Application No. 63/451,625, filed on Mar. 12, 2023, which is incorporated herein by reference in its entirety.

### BACKGROUND

Cervical cancer is a major health concern among women. Worldwide in 2018, there were approximately 570.000 cases of cervical cancer, and about 311,000 deaths due to the disease. (Arbyn et al., 2020). It was the fourth most common cancer in women, behind only breast cancer, colorectal cancer, and lung cancer. (Id.). In the U.S., the number of estimated new cervical cancer cases for 2023 is 13,360, which will account for over 12% of estimated new cases for gynecological cancers in women (Siegel et al., 2022).

Early diagnosis for cervical cancer has a significant impact on survival rate. Under the revised 2018 International Federation of Gynecology and Obstetrics (FIGO) cervical cancer staging schema, five-year survival of patients diagnosed with cervical cancer at Stage I is 85.6% (Wright et al., 2019) But the five-year survival rate drops to 56.1% for patients with Stage II cervical cancer, to 39.3% for patients with Stage III cervical cancer, and to 24% for patients with Stage IV cervical cancer (id.).

It is recommended that women undergo repeated Papanicolaou (Pap) tests to ensure that cervical cancer is detected early. However, cervical cytology is expensive, and the procedure requires Board-certified pathologists to review one by one and interpret the results from Pap smears.

Therefore, there is an urgent unmet clinical need to improve the detection and diagnosis of cervical cancer.

### SUMMARY OF THE INVENTION

Some of the main aspects of the present invention are summarized below. Additional aspects are described in the Detailed Description of the Invention, Examples, Drawings, and Claims sections of this disclosure. The description in each section of this disclosure is intended to be read in conjunction with the other sections. Furthermore, the various embodiments described in each section of this disclosure can be combined in various different ways, and all such combinations are intended to fall within the scope of the present invention.

One aspect of the invention relates to a method of evaluating a subject for cervical cancer, the method comprising determining in a biological sample from the subject a concentration of one or more proteins selected from Table 1. In some embodiments, the method further comprises applying a classifier to the concentration of the one or more proteins that identifies whether the concentration of the one or more proteins is indicative of the subject having cervical cancer. In some embodiments, the method further comprises administering a treatment to the subject.

Another aspect of the invention relates to a method of treating cervical cancer in a subject, comprising acquiring results from a method of evaluating a subject for cervical cancer as described herein, and administering a treatment to the subject.

Another aspect of the invention relates to a method of detecting cervical cancer in a subject, the method comprising determining in a biological sample from the subject a concentration of one or more proteins selected from Table 1; and applying a classifier to the concentration of the one or more proteins that identifies whether the concentration of the one or more proteins is indicative that cervical cancer is detected.

Yet another aspect of the invention relates to a method of treating cervical cancer in a subject, comprising acquiring results from a method of detecting cervical cancer in a subject as described herein, and administering a treatment to the subject.

Another aspect of the invention relates to a method of treating cervical cancer in a subject in whom cervical cancer was detected, the method comprising administering a treatment for cervical cancer to the subject, in which cervical cancer was detected in the subject by a method comprising determining in a biological sample from the subject a concentration of one or more proteins selected from Table 1; and applying a classifier to the concentration of the one or more proteins that identifies whether the concentration of the one or more proteins is indicative that cervical cancer is detected.

In some embodiments, the cervical cancer is early-stage.

In some embodiments, the subject is asymptomatic of cervical cancer.

Another aspect of the invention relates to a method of evaluating a treatment for cervical cancer in a subject, the method comprising administering a treatment for cervical cancer, and determining in a biological sample from the subject a concentration of one or more proteins selected from Table 1.

Another aspect of the invention relates to a method of evaluating the efficacy of a treatment for cervical cancer in a subject, the method comprising administering a treatment for cervical cancer to the subject, and determining in a biological sample from the subject a concentration of one or more proteins selected from Table 1.

Another aspect of the invention relates to a method of treating cervical cancer in a subject, the method comprising administering a treatment for cervical cancer to the subject, and determining in a biological sample from the subject a concentration of one or more proteins to evaluate the efficacy of the treatment, wherein the one or more proteins are selected from Table 1.

Another aspect of the invention relates to a method of adjusting a treatment for cervical cancer in a subject, the method comprising administering a treatment for cervical cancer to the subject, and determining in a biological sample from the subject a concentration of one or more proteins, wherein the one or more proteins are selected Table 1.

Yet another aspect of the invention relates to a method of treating cervical cancer in a subject, the method comprising administering a treatment for cervical cancer to the subject, and determining in a biological sample from the subject a concentration of one or more proteins to evaluate whether the treatment requires adjustment, wherein the one or more proteins are selected from Table 1.

Another aspect of the invention relates to a method of monitoring for cervical cancer recurrence in a subject, the method comprising administering a treatment for cervical cancer to the subject, and determining in a biological sample from the subject a concentration of one or more proteins to evaluate whether the treatment requires adjustment, wherein the one or more proteins are selected from Table 1. In some embodiments, the method further comprises administering an adjusted treatment when it is determined that the treatment requires adjustment.

Another aspect of the invention relates to a method of treating cervical cancer in a subject, the method comprising administering a treatment for cervical cancer to the subject, and determining in a biological sample from the subject a concentration of one or more proteins to evaluate whether cancer is recurring, wherein the one or more proteins are selected from Table 1. In some embodiments, the method further comprises administering a second treatment when it is determined that the cancer is recurring.

In some embodiments, the biological sample is selected from a plasma sample, serum sample, saliva sample, cerebrospinal fluid (CSF) sample, sweat sample, urine sample, or tear sample. In preferred embodiments, the biological sample is a urine sample.

In some embodiments, the one or more proteins are selected from Table 2.

In some embodiments, the one or more proteins are selected from Table 3.

In some embodiments, the one or more proteins are selected from Table 4. In certain embodiments, the one or more proteins are each protein from Table 4.

Another aspect of the invention relates to a method of measuring amounts of proteins in a subject, the method comprising determining individual amounts of one or more proteins selected from Table 1.

Yet another aspect of the invention relates to a method of measuring amounts of proteins in a subject, the method comprising determining individual amounts of one or more proteins selected from Table 2.

Another aspect of the invention relates to a method of measuring amounts of proteins in a subject, the method comprising determining individual amounts of one or more proteins selected from Table 3.

Another aspect of the invention relates to a method of measuring amounts of proteins in a subject, the method comprising determining individual amounts of one or more proteins selected from Table 4. In certain embodiments, the method comprises determining individual amounts of each protein from Table 4.

Another aspect of the invention relates to a kit comprising one or more components that can be used to perform assays for detecting one or more proteins of Table 1, or one or more proteins of Table 2, or one or more proteins of Table 3, or one or more proteins of Table 4. In some embodiments, the one or more proteins are selected from Table 2. In certain embodiments, the one or more proteins are selected from Table 3. In some embodiments, the one or more proteins are selected from Table 4. In certain embodiments, the one or more proteins are each protein from Table 4.

### BRIEF DESCRIPTION OF THE FIGURES

FIG. 1 shows accuracy, measured as area-under-the-curve (AUC) of a receiver operating characteristic (ROC) curve, of detecting cervical cancer in a subject using random combinations of two to 20 proteins selected from Table 1, as described in the Example. The process of selecting the random combinations of each number of proteins (two proteins, three proteins, etc.) was performed for 1000 iterations.

FIG. 2 shows an ROC curve generated by application of a classifier, which depicts the high diagnostic utility of detecting cervical cancer in a subject using the panel of 20 proteins listed in Table 2, as described in the Example.

FIG. 3 shows an ROC curve generated by application of a classifier, which depicts the high diagnostic utility of detecting cervical cancer in a subject using the panel of 37 proteins listed in Table 3, as described in the Example.

FIG. 4 shows ROC curves generated by application of a classifier, which depicts the high diagnostic utility of detecting cervical cancer in a subject using each of the seven proteins listed in Table 4, both individually (solid lines) and in combination (starred line), as described in the Example.

### DETAILED DESCRIPTION OF THE INVENTION

The practice of the present invention can employ, unless otherwise indicated, conventional techniques of proteomics, bioinformatics, oncology, and pharmacology, which are within the skill of the art.

In order that the present invention can be more readily understood, certain terms are first defined. Additional definitions are set forth throughout the disclosure. Unless defined otherwise, all technical and scientific terms used herein have the same meaning as commonly understood by one of ordinary skill in the art to which this invention is related.

Any headings provided herein are not limitations of the various aspects or embodiments of the invention, which can be had by reference to the specification as a whole. Accordingly, the terms defined immediately below are more fully defined by reference to the specification in its entirety.

All references cited in this disclosure are hereby incorporated by reference in their entireties. In addition, any manufacturers' instructions or catalogues for any products cited or mentioned herein are incorporated by reference. Documents incorporated by reference into this text, or any teachings therein, can be used in the practice of the present invention. Documents incorporated by reference into this text are not admitted to be prior art.

### Definitions

The phraseology or terminology in this disclosure is for the purpose of description and not of limitation, such that the terminology or phraseology of the present specification is to be interpreted by the skilled artisan in light of the teachings and guidance.

As used in this specification and the appended claims, the singular forms “a,” “an,” and “the” include plural referents, unless the context clearly dictates otherwise. The terms “a” (or “an”) as well as the terms “one or more” and “at least one” can be used interchangeably.

Furthermore, “and/or” is to be taken as specific disclosure of each of the two specified features or components with or without the other. Thus, the term “and/or” as used in a phrase such as “A and/or B” is intended to include A and B, A or B, A (alone), and B (alone). Likewise, the term “and/or” as used in a phrase such as “A, B, and/or C” is intended to include A, B, and C; A, B, or C; A or B; A or C; B or C; A and B; A and C; B and C; A (alone); B (alone); and C (alone).

Units, prefixes, and symbols are denoted in their Systeme International de Unites (SI) accepted form. Numeric ranges are inclusive of the numbers defining the range, and any individual value provided herein can serve as an endpoint for a range that includes other individual values provided herein. For example, a set of values such as 1, 2, 3, 8, 9, and 10 is also a disclosure of a range of numbers from 1-10. Where a numeric term is preceded by “about,” the term includes the stated number and values±10% of the stated number. The headings provided herein are not limitations of the various aspects or embodiments of the invention, which can be had by reference to the specification as a whole. Accordingly, the terms defined immediately below are more fully defined by reference to the specification in its entirety.

Wherever embodiments are described with the language “comprising,” otherwise analogous embodiments described in terms of “consisting of” and/or “consisting essentially of” are included.

An “effective amount” of a composition as disclosed herein is an amount sufficient to carry out a specifically stated purpose. An “effective amount” can be determined empirically and in a routine manner, in relation to the stated purpose, route of administration, and dosage form.

The term “subject” or “individual” or “patient” means any subject, preferably a mammalian subject, for whom diagnosis, prognosis, or therapy is desired. Mammalian subjects include humans, domestic animals, farm animals, sports animals, and zoo animals including, e.g., humans, non-human primates, dogs, cats, guinea pigs, rabbits, rats, mice, horses, cattle, and so on.

The term “early-stage” in the context of cancer (e.g., “early-stage cancer” or cancer that “is early-stage”) refers generally to a level of advancement of the cancer prior to the cancer spreading to lymph nodes or tissues that are distant from the tissue of origin. In some embodiments, an early-stage cancer can refer to a cancer that is a Stage 0, Stage I, or Stage II cancer, based on the stage classification known in the art that grades cancer from Stage 0 (e.g., carcinoma in situ, where the cancer is still only in the layer of cells where it started and has not advanced farther), through Stages I-III (e.g., cancer is present—the higher the number, the larger the tumor and the more it has spread into nearby tissues), and to Stage IV (e.g., the cancer has spread to distant parts of the body). In some embodiments, this stage classification incorporates the TNM System, which evaluates the cancer based on the size and extent of the main tumor (“T”), the number of nearby lymph nodes that have cancer (“N”), and the extent to which the cancer has metastasized (“M”).

The term “symptomatic” means to exhibit one or more signs or features that are regarded as indicative, or are known to be associated with, a disease or condition. A subject may be considered as “symptomatic” of cancer based on symptoms that are known in the art to be associated with cancer in general or for specific types of cancer. Examples include, but are not limited to, fatigue; lump or area of thickening that can be felt under the skin; weight changes, including unintended loss or gain; skin changes, such as yellowing, darkening, or redness of the skin, sores that will not heal, or changes to existing moles: changes in bowel or bladder habits; persistent cough or trouble breathing; difficulty swallowing; hoarseness; persistent indigestion or discomfort after eating; persistent, unexplained muscle or joint pain; persistent, unexplained fevers or night sweats; and unexplained bleeding or bruising. Symptoms that can occur with cervical cancer in particular include, but are not limited to, abnormal vaginal bleeding, such as bleeding after vaginal sex, bleeding after menopause, bleeding and spotting between periods, having (menstrual) periods that are longer or heavier than usual, or bleeding after douching; unusual discharge from the vagina that may contain some blood and may occur between periods or after menopause; pain during sex; pain in the pelvic region; swelling of the legs; problems urinating or having a bowel movement; and blood in the urine.

A subject may be considered as “suspected of having a cancer” due to the presence of symptoms, i.e., the subject is symptomatic; genetic markers (e.g., mutations in BRCA1, BRCA2, RAS, BRAF, etc.); patient's habits or medical history; patient's family medical history; examination or tests known in the art for which the outcome is associated with cancer or risk of cancer, etc.

The term “asymptomatic” means to not exhibit any signs or features that are regarded as indicative, or are known to be associated with, a disease or condition.

Terms such as “treating” or “treatment” or “to treat” or “alleviating” or “to alleviate” refer to therapeutic measures that cure, slow down, lessen symptoms of, and/or halt progression of a diagnosed pathologic condition or disorder. Thus, those in need of treatment include those already with the disorder. In certain embodiments, a subject is successfully “treated” for a disease or disorder if the patient shows, e.g., total, partial, or transient alleviation or elimination of symptoms associated with the disease or disorder.

The term “ROC” or “ROC curve” is used to refer to a receiver operator characteristic curve. A ROC curve can be a graphical representation of the performance of a classifier system. For any given method, a ROC can be generated by plotting the sensitivity against the specificity. The sensitivity and specificity of a method for detecting the presence of a cancer or a specific type of cancer can be determined at various concentrations of proteins in a sample from the subject. The AUC of a ROC curve is a metric that can provide a measure of diagnostic utility of a method, taking into account both the sensitivity and specificity of the method. The AUC can range from 0.5 to 1.0, where a value closer to 0.5 can indicate that the method has limited diagnostic utility (e.g., lower sensitivity and/or specificity) and a value closer to 1.0 indicates the method has greater diagnostic utility (e.g., higher sensitivity and/or specificity).

The term “third party” means a person or group different from the two persons or groups primarily involved. For example, in a multi-step method involving a subject, a third party can be a person/group other than the subject and the person/group primarily responsible for the performance of the steps. In such an example, a third party may perform one of the steps in the method. As another example, in a treatment method involving administration of a treatment to a subject, a third party may be a person/group other than the subject and the person/group administering the treatment.

The term “cancer recurrence” refers to a return of cancer after a period of remission. The cancer can reappear in the same, or close to, the place that it was previously found (local recurrence); in the lymph nodes and tissue located in the vicinity of the original cancer (regional recurrence); or in areas farther away from the original cancer (distant recurrence).

### Methods of the Invention

The present invention involves the use of proteins in the detection of evaluation of cervical cancer in subjects (also referred to herein as “cervical cancer detection proteins”). Such use can be applied in methods of evaluating a subject for cervical cancer, methods of treating subjects for cervical cancer, among others.

The proteins can be used to detect or evaluate cervical cancer based on a biological sample from the subject. The biological sample may be any biological sample capable of being obtained from the subject, and encompass fluids, solids, tissues, and gases. In some embodiments, the sample may be a blood product, such as plasma, serum and the like. In some embodiments, the sample may be a urine sample, saliva sample, CSF sample, sweat sample, or tear sample.

In preferred embodiments, the biological sample is advantageously a urine sample. Compared to blood or plasma samples, there is no homeostasis mechanism in urine that can regulate the presence of proteins in the course of maintaining relatively constant physical/chemical properties within the body (Jing, 2018). It is possible that potential biomarkers may be cleared from plasma or blood by the inherent homeostasis mechanism in order to avoid possible damage or interference to the body (id.). On the other hand, the waste materials in the urine are the cleared objects of the blood homeostasis mechanism and therefore may better reflect changes that are produced in vivo by the presence of a disease such as cervical cancer and that would not be cleared by any homeostasis mechanism (id.). In addition, urine collection is less traumatic to the body and involves no infliction of pain, is safer and less costly, and is easier and simpler to store (id.).

An aspect of the present invention relates to a method of evaluating a subject for a cancer that is associated with the cervix; or a method of evaluating a subject for cervical cancer. The method comprises determining in a biological sample from the subject a concentration of one or more proteins selected from Table 1.

In preferred embodiments, the sample is already separated/obtained/collected from the subject at the time of the evaluation. In some embodiments, the sample is separated from the subject at home and/or by the subject prior to the evaluation.

In embodiments of the invention, the method identifies whether the subject has cervical cancer. The method may further comprise applying a classifier to the concentration of the one or more cervical cancer detection proteins. The classifier identifies whether the concentration of the one or more cervical cancer detection proteins is indicative that the subject has cervical cancer.

In embodiments of the invention, the methods of evaluating a subject further comprise administering a treatment. In some embodiments, the treatment is administered when it is determined that the subject has cervical cancer.

To this end, an aspect of the present invention relates to a method of treating cervical cancer in a subject, comprising (a) acquiring results from methods of evaluating a subject for cervical cancer as described herein, and (b) administering a treatment to the subject. In some embodiments, the results from methods of evaluating a subject for cervical cancer are provided by a third party. In some embodiments, the treatment is responsive to the results, e.g., responsive to having cervical cancer.

Another aspect of the present invention relates to a method of treating cervical cancer in a subject, in which the method comprises (a) acquiring results from an evaluation of the subject that determined the subject has cervical cancer; (b) administering a treatment to the subject. e.g., a treatment for cervical cancer, in which the evaluation comprises: (I) determining in a biological sample from the subject a concentration of one or more proteins selected from Table 1, and (II) applying a classifier to the concentration of the one or more proteins to identify whether the subject has cervical cancer. In some embodiments, the results in (a) are acquired from a third party.

An aspect of the present invention relates to a method of detecting cervical cancer in a subject, the method comprising determining in a biological sample from the subject a concentration of one or more proteins selected from Table 1, and applying a classifier to the concentration of the one or more proteins that identifies whether the concentration of the one or more proteins is indicative that cervical cancer is detected.

In embodiments of the invention, the method of detecting cervical cancer in a subject further comprises administering a treatment. In some embodiments, the treatment is administered when cervical cancer is detected.

To this end, an aspect of the present invention relates to a method of treating cervical cancer in a subject, comprising (a) acquiring results from a method of detecting cervical cancer in a subject as described herein, and (b) administering a treatment to the subject. In some embodiments, the results from the method of detecting cervical cancer in a subject are provided by a third party. In some embodiments, the treatment is responsive to the results, e.g., responsive to cervical cancer being detected.

An aspect of the invention relates to a method of treating cervical cancer in a subject in whom cervical cancer was detected, the method comprising administering a treatment for the cervical cancer; in which the cervical cancer had been detected in the subject by a method comprising determining in a biological sample from the subject a concentration of one or more proteins selected from Table 1, and applying a classifier to the concentration of the one or more proteins to identify whether the subject has cervical cancer. In some embodiments, the method of detecting the cervical cancer was performed by a third party.

Yet another aspect of the present invention relates to a method of treating cervical cancer in a subject, in which the method comprises (a) determining in a biological sample from the subject a concentration of one or more proteins selected from Table 1; (b) applying a classifier to the concentration of the one or more proteins to identify that the subject has cervical cancer; and (c) administering a treatment to the subject, e.g., a treatment for cervical cancer.

Another aspect of the present invention relates to a method of treating cancer in a patient who has been or was determined to have cervical cancer, comprising administering a treatment for cervical cancer to the patient, in which the patient was determined to have cervical cancer by a method comprising (a) determining in a biological sample from the subject a concentration of one or more proteins selected from Table 1, and (b) applying a classifier to the concentration of the one or more proteins. The classifier identifies whether the concentration of the one or more proteins is indicative that the subject has cervical cancer.

In some embodiments, the subject is asymptomatic for cervical cancer. In some embodiments, the methods may be performed as part of, or may be included within, or may overlap with, a screening for cervical cancer in the subject. In some embodiments, the subject is undergoing a screen for cervical cancer.

In some embodiments, the subject is suspected of having cervical cancer, such as symptomatic of having cervical cancer.

In addition, an aspect of the present invention relates to a method of evaluating a treatment for cervical cancer in a subject. The method comprises (a) administering a treatment for cervical cancer, and (b) determining in a biological sample from the subject a concentration of one or more proteins selected from Table 1. In preferred embodiments, the sample is already separated/obtained from the subject at the time of performing (b). In some embodiments, administration of the treatment in (a) may be performed by a third party. In other embodiments, determining the concentration of the one or more proteins in (b) may be performed by a third party.

In embodiments of the invention, the one or more proteins identifies whether the subject has cervical cancer after treatment. Thus, the method may further comprise applying a classifier to the concentration of the one or more proteins that identifies whether the concentration of the one or more proteins is indicative of the subject having cervical cancer.

The treatment may be any known treatment for cancer as known in the art and as described herein. The administration of the treatment in (a) may comprise a single administration or occurrence of a therapy, or may comprise multiple administrations or occurrences of a therapy.

The determination in a biological sample from the subject a concentration of one or more proteins in (b) may be performed more than once. The determination may overlap with the administration of the treatment in (a) or may occur after the administration of the treatment in (a).

In embodiments in which determination in a biological sample from the subject a concentration of one or more proteins in (b) is occurring after the administration of the treatment in (a), the determination may occur immediately after the administration of the treatment or a period of time after the administration of the treatment. The period of time may be one day or more, or one week or more, or one month or more, or one year or more: including one day, or two days, or three days, or four days, or five days, or six days, or about one week, or about two weeks, or about three weeks, or about four weeks, or about five weeks, or about six weeks, or about seven weeks, or about eight weeks, or about nine weeks, or about ten weeks, or about 11 weeks, or about 12 weeks, or about one month, or about two months, or about three months, or about four months, or about five months, or about six months, or about seven months, or about eight months, or about nine months, or about ten months, or about 11 months, or about 12 months, or about one year, or about two years, or about three years, or about four years, or about five years, or about six years, or about seven years, or about eight years, or about nine years, or about ten years, or about 11 years, or about 12 years, or about 13 years, or about 14 years, or about 15 years, or about 16 years, or about 17 years, or about 18 years, or about 19 years, or about 20 years, or about 21 years, or about 22 years, or about 23 years, or about 24 years, or about 25 years, or about 26 years, or about 27 years, or about 28 years, or about 29 years, or about 30 years, or more; including any ranges formed with these time periods as endpoints, for examples about 4 weeks to about 13 years, about 7 months to about 3 years, etc.

In some embodiments, the presence of cervical cancer after treatment may be indicative that the treatment was not effective. Thus, another aspect of the invention is a method of evaluating the efficacy of a cervical cancer treatment, comprising (a) administering a treatment for cervical cancer, and (b) determining in a biological sample from the subject a concentration of one or more proteins, as described herein. Yet another aspect is a method of treatment, comprising (a) administering a treatment for cervical cancer, and (b) determining in a biological sample from the subject a concentration of one or more proteins, as described herein, to evaluate whether the treatment was effective.

In some embodiments, the presence of cervical cancer after treatment may be indicative that the treatment requires adjustment. Thus, another aspect of the invention is a method of adjusting a treatment for cervical cancer, comprising (a) administering a treatment for cervical cancer, and (b) determining in a biological sample from the subject a concentration of one or more proteins, as described herein, to evaluate whether the treatment requires adjustment; such method may further comprise administering a second treatment. The second treatment may be different from the original treatment, for example, a different therapy or different dosage of the same therapy.

In some embodiments, the presence of cervical cancer after treatment may be indicative of cancer recurrence. Thus, another aspect of the invention is a method of monitoring for cervical cancer recurrence, comprising (a) administering a treatment for cervical cancer, and (b) determining in a biological sample from the subject a concentration of one or more proteins, as described herein. Yet another aspect is a method of treatment, comprising (a) administering a treatment for cervical cancer, and (b) determining in a biological sample from the subject a concentration of one or more proteins, as described herein, to evaluate cancer recurrence. In some embodiments, the method may further comprise administering a second treatment when it is determined that the cervical cancer is recurring. The second treatment may be different from the original treatment, for example, a different therapy or different dosage of the same therapy.

An aspect of the present invention relates to a method of measuring amounts of proteins in a subject, the method comprising determining individual amounts of one or more proteins selected from Table 1. In some embodiments, the individual amounts of the one or more proteins is determined in a biological sample from the subject.

In some embodiments, the biological sample is a plasma sample, serum sample, saliva sample, CSF sample, sweat sample, urine sample, or tear sample. In preferred embodiments, the biological sample is a urine sample.

In embodiments of the invention, the methods may further comprise obtaining or collecting a biological sample from the subject before determining the concentration of one or more proteins in the biological sample. The collection of the biological sample may be performed in a home (e.g., the home of the subject) or at a medical facility (e.g., doctor's office, hospital, urgent care center, etc.).

In some embodiments, the determination of the concentration of one or more proteins in the biological sample may be performed in a home (e.g., the home of the subject) or at a medical facility (e.g., doctor's office, hospital, urgent care center, etc.).

In some embodiments of the invention, the one or more proteins may be selected from Table 2. In some embodiments of the invention, the one or more proteins may be each protein of Table 2.

In some embodiments of the invention, the one or more proteins may be selected from Table 3. In some embodiments of the invention, the one or more proteins may be each protein of Table 3.

In some embodiments of the invention, the one or more proteins may be selected from Table 4. In some embodiments of the invention, the one or more proteins may be each protein of Table 4.

In some embodiments of the present invention, for any of the cervical cancer detection proteins, the methods may comprise determining the concentration of two or more, or three or more, or four or more, or five or more, or six or more, or seven or more, or eight or more, or nine or more, or ten or more, or about 15 or more, or about 20 or more, or about 25 or more, or about 30 or more, or about 35 or more, or about 40 proteins or more, or about 40 or more, or about 45 proteins or more, or about 50 proteins or more, or about 55 proteins or more, proteins: including any number of proteins chosen from two, three, four, five, six, seven, eight, nine, ten, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, or 60; and including any ranges thereof, for example, about two to 60 proteins, or about two to 55 proteins, or about two to 50 proteins, or about two to 45 proteins, or about two to 40 proteins, or about two to 35 proteins, or about two to 30 proteins, or about two to 25 proteins, or about two to 20 proteins, or about two to 15 proteins, or about two to ten proteins, or about two to nine proteins, or about two to eight proteins, or about two to seven proteins, or about two to six proteins, or about two to five proteins, or about two to four proteins, or about two or three proteins, or about three to 60 proteins, or about three to 55 proteins, or about three to 50 proteins, or about three to 45 proteins, or about three to 40 proteins, or about three to 35 proteins, or about three to 30 proteins, or about three to 25 proteins, or about three to 20 proteins, or about three to 15 proteins, or about three to ten proteins, or about three to nine proteins, or about three to eight proteins, or about three to seven proteins, or about three to six proteins, or about three to five proteins, or about three or four proteins, or about five to 60 proteins, or about five to 55 proteins, or about five to 50 proteins, or about five to 45 proteins, or about five to 40 proteins, or about five to 35 proteins, or about five to 30 proteins, or about five to 25 proteins, or about five to 20 proteins, or about five to 15 proteins, or about five to ten proteins, or about five to nine proteins, or about five to eight proteins, or about five to seven proteins, or about five or six proteins, or about ten to 60 proteins, or about ten to 55 proteins, or about ten to 50 proteins, or about ten to 45 proteins, or about ten to 40 proteins, or about ten to 35 proteins, or about ten to 30 proteins, or about ten to 25 proteins, or about ten to 20 proteins, or about ten to 15 proteins, or about 15 to 60 proteins, or about 15 to 55 proteins, or about 15 to 50 proteins, or about 15 to 45 proteins, or about 15 to 40 proteins, or about 15 to 35 proteins, or about 15 to 30 proteins, or about 15 to 25 proteins, or about 15 to 20 proteins, or about 20 to 60 proteins, or about 20 to 55 proteins, or about 20 to 50 proteins, or about 20 to 45 proteins, or about 20 to 40 proteins, or about 20 to 35 proteins, or about 20 to 30 proteins, or about 20 to 25 proteins, or about 25 to 60 proteins, or about 25 to 55 proteins, or about 25 to 50 proteins, or about 25 to 45 proteins, or about 25 to 40 proteins, or about 25 to 30 proteins, or about 30 to 60 proteins, or about 30 to 55 proteins, or about 30 to 50 proteins, or about 30 to 45 proteins, or about 30 to 40 proteins, or about 30 to 35 proteins, or about 35 to 60 proteins, or about 35 to 55 proteins, or about 35 to 50 proteins, or about 35 to 45 proteins, or about 35 to 40 proteins, or about 40 to 60 proteins, or about 40 to 55 proteins, or about 40 to 50 proteins, or about 40 to 45 proteins, or about 45 to 60 proteins, or about 45 to 55 proteins, or about 45 to 50 proteins, or about 50 to 60 proteins, or about 50 to 55 proteins, or about 55 to 60 proteins.

In some embodiments, the methods may comprise determining the concentration of each protein of Table 1. In certain embodiments, the methods may comprise determining the concentration of each protein of Table 2. In certain embodiments, the methods may comprise determining the concentration of each protein of Table 3. In certain embodiments, the methods may comprise determining the concentration of each protein of Table 4.

In some embodiments, the number of proteins for which the concentration is determined may be sufficient to achieve an AUC of a ROC curve of at least about 0.6. In certain embodiments, the number of proteins for which the concentration is determined may be sufficient to achieve an AUC of a ROC curve of at least about 0.7, or at least about 0.8, or at least about 0.9.

In embodiments of the invention, the cervical cancer is early-stage. In some embodiments, the cervical cancer is stage I. In some embodiments, the cervical cancer is stage II.

In some embodiments, the cervical cancer is stage III. In some embodiments, the cervical cancer is stage IV. In some embodiments, the cervical cancer is stage V.

The treatment administered to the subjects according to the methods described herein may be treatments known in the art. Examples of such treatments include, but are not limited to, surgery, radiation therapy, chemotherapy, immunotherapy, targeted therapy, and any combination thereof. Examples of surgery may include, but are not limited to, hysterectomy, such as a simple hysterectomy (removal of the uterus and cervix) or radical hysterectomy (removal of the entire uterus, tissues next to the uterus, and the upper part of the vagina); trachelectomy (removal of the cervix and the upper part of the vagina, but not the body of the uterus); lymph node dissection (removal of lymph nodes, such as those in the pelvis or around the aorta); pelvic exenteration (removal of the same tissues as a radical hysterectomy and of lymph nodes, as well as potentially other tissues such as the bladder, vagina, rectum, and part of the colon); and any combination thereof. Examples of radiation therapy include, but are not limited to, brachytherapy, external beam radiation therapy, and a combination thereof. Examples of chemotherapy include, but are not limited to, paclitaxel, carboplatin, topotecan, cisplatin, docetaxel, ifosfamide, fluorouracil, irinotecan, gemcitabine, mitomycin, and any combination thereof. Examples of targeted therapy include, but are not limited to, tisotumab vedotin-tftv, bevacizumab, and a combination thereof. Examples of immunotherapy include, but are not limited to, immune checkpoint inhibitors such as pembrolizumab.

In certain embodiments, a cancer patient subjected to a method of the invention is successfully treated if the patient's survival is longer than the median survival of patients having cervical cancer. Survival can be overall survival, i.e., length of time a patient lives, or progression-free survival, i.e., length of time a patient is treated without progression of the disease. Survival can be measured from the date of diagnosis or from the date that treatment commences. Overall survival, median overall survival, progression-free survival, and median progression-free survival can be determined by methods known in the art and/or by those described herein.

In certain embodiments a patient with cervical cancer subjected to a method of the invention is successfully treated if the patient has an improved response to the anti-cancer therapy compared with a patient having cervical cancer who has not been subjected to a method of the invention. For example, treatment of cervical cancer would be successful in a subject treated by the methods of the invention if the subject has an improved response compared to the median response of patients who have not been treated by the methods of the invention. Response to anti-cancer treatment can be measured by known methods appropriate to the cancer type, for instance, using Response Evaluation Criteria in Solid Tumors (RECIST). Patients evaluated using RECIST can have a complete response (CR), a partial response (PR), stable disease (SD), or progressive disease (PD). An improved response can also be assessed by other criteria, for example, duration of response, reduction in tumor volume, minimum residual disease (MRD), and the like.

### Protein Concentration Measurement and Application of Classifiers

The concentration of proteins in the sample may be measured using protein quantitation techniques known in the art. Such techniques include, but are not limited to, enzyme-linked immunosorbent assays, chemiluminescence immunoassays, immunohistochemistry, liquid-bead immunoassays, mass spectrometry, aptamer-based assays, reverse phase protein arrays, proximity extension assay (PEA), and a combination thereof.

In the methods described herein, the concentration of the two or more proteins are used and combined with mathematical, statistical, and machine-learning methods to create secondary features. One or more proteins with and without secondary features and baseline features, including age, sex, race and ethnicity, past medical history, family history, patient's lab values, comorbidities, and concomitant medications, are used in one or more predictive models to calculate a score.

Machine learning and statistical analyses techniques used to generate features and the final score for the cancer are included but not limited to the following concepts and methods: Supervised learning concepts may include AODE; Artificial neural network, such as Backpropagation, Auto encoders, Hopfield networks. Boltzmann machines, Restricted Boltzmann Machines, and Spiking neural networks; Bayesian statistics, such as Bayesian network and Bayesian knowledge base; Case-based reasoning; Gaussian process regression; Gene expression programming; Group method of data handling (GMDH); Inductive logic programming; Instance-based learning; Lazy learning: Learning Automata; Learning Vector Quantization; Logistic Model Tree; Minimum message length (decision trees, decision graphs, etc.), such as Nearest Neighbor Algorithm and Analogical modeling; Probably approximately correct learning (PAC) learning: Ripple down rules, a knowledge acquisition methodology; Symbolic machine learning algorithms; Support vector machines; Random Forests; Ensembles of classifiers, such as Bootstrap aggregating (bagging) and Boosting (meta-algorithm); Ordinal classification; Information fuzzy networks (IFN): Conditional Random Field; ANOVA; Linear classifiers, such as Fisher's linear discriminant, Linear regression. Logistic regression, Multinomial logistic regression, I Bayes classifier, Perceptron, Support vector machines; Quadratic classifiers; k-nearest neighbor; Boosting; Decision trees, such as C4.5, Random forests, ID3, CART, SLIQ SPRINT; Bayesian networks, sucINaive Bayes; and Hidden Markov models. Unsupervised learning concepts may include: Expectation-maximization algorithm; Vector Quantization; Generative topographic map: Information bottleneck method; Artificial neural network, such as Self-organizing map; Association rule learning, such as Apriori algorithm, Eclat algorithm, and FP growth algorithm; Hierarchical clusterings such as Single linkage clustering and Conceptual clustering; Cluster analysis, such as K-means algorithm, Fuzzy clustering, DBSCAN, and OPTICS algorithm; and Outlier Detection, such as Local Outlier Factor. Semi-supervised learning concepts may include: Generative models; Low-density separation; Graph-based methods, and Co-training. Reinforcement learning concepts may include Temporal difference learning; Q-learning, Learning Automata, and SARSA. Deep learning concepts may include Deep belief networks; Deep Boltzmann machines: Deep Convolutional neural networks: Deep Recurrent neural networks; and Hierarchical temporal memory.

For concentrations obtained from detection proteins, one or more features are fed into one or more computation models. The classifiers are used to calculate a score for the patient. The scores of different classifiers are combined to identify the patient as having the specific cancer or not. The computational model may use one or more proteins or secondary features with and without baseline features that could generate a (ROC curve greater than or equal to 0.6. This step determines if the sample indicates the presence of the cancer.

Protein concentrations and/or secondary features are fed into one or more predictive models. The features could be similar or different from what was used in determining cancer status. The classifiers are used to calculate a score for the patient for cervical cancer. The predictive models use the proteins or derived secondary features that could generate a ROC curve greater than or equal to 0.6.

Generally, machine learning algorithms are used to construct models that accurately assign class labels to examples based on the input features that describe the example.

Embodiments of the present disclosure can be further defined by reference to the following non-limiting examples. It will be apparent to those skilled in the art that many modifications, both to materials and methods, can be practiced without departing from the scope of the present disclosure.

### Kit

An aspect of the present invention relates to a kit for use in detecting one or more cervical cancer detection proteins, i.e., one or more proteins of Table 1, or one or more proteins of Table 2, or one or more proteins of Table 3, or one or more proteins of Table 4, or each protein of Table 4, which can be used to perform the methods described herein. The kit may comprise one or more components that can be used to perform assays such as enzyme-linked immunosorbent assays, chemiluminescence immunoassays, immunohistochemistry, liquid-bead immunoassays, mass spectrometry, aptamer-based assays, reverse phase protein arrays, PEA, or a combination thereof. Such components include, but are not limited to, antibodies or antigen binding fragments thereof that bind one or more proteins of Table 1, or one or more proteins of Table 2, or one or more proteins of Table 3, or one or more proteins of Table 4, or each protein of Table 4.

In some embodiments, the kit comprises antibodies or antigen binding fragments thereof that bind two or more, or three or more, or four or more, or five or more, or six or more, or seven or more, or eight or more, or nine or more, or ten or more, or about 15 or more, or about 20 or more, or about 25 or more, or about 30 or more, or about 35 or more, or about 40 proteins or more, or about 40 or more, or about 45 proteins or more, or about 50 proteins or more, or about 55 proteins or more, proteins; including any number of proteins chosen from two, three, four, five, six, seven, eight, nine, ten, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51, 52, 53, 54, 55, 56, 57, 58, 59, or 60; and including any ranges thereof, for example, about two to 60 proteins, or about two to 55 proteins, or about two to 50 proteins, or about two to 45 proteins, or about two to 40 proteins, or about two to 35 proteins, or about two to 30 proteins, or about two to 25 proteins, or about two to 20 proteins, or about two to 15 proteins, or about two to ten proteins, or about two to nine proteins, or about two to eight proteins, or about two to seven proteins, or about two to six proteins, or about two to five proteins, or about two to four proteins, or about two or three proteins, or about three to 60 proteins, or about three to 55 proteins, or about three to 50 proteins, or about three to 45 proteins, or about three to 40 proteins, or about three to 35 proteins, or about three to 30 proteins, or about three to 25 proteins, or about three to 20 proteins, or about three to 15 proteins, or about three to ten proteins, or about three to nine proteins, or about three to eight proteins, or about three to seven proteins, or about three to six proteins, or about three to five proteins, or about three or four proteins, or about five to 60 proteins, or about five to 55 proteins, or about five to 50 proteins, or about five to 45 proteins, or about five to 40 proteins, or about five to 35 proteins, or about five to 30 proteins, or about five to 25 proteins, or about five to 20 proteins, or about five to 15 proteins, or about five to ten proteins, or about five to nine proteins, or about five to eight proteins, or about five to seven proteins, or about five or six proteins, or about ten to 60 proteins, or about ten to 55 proteins, or about ten to 50 proteins, or about ten to 45 proteins, or about ten to 40 proteins, or about ten to 35 proteins, or about ten to 30 proteins, or about ten to 25 proteins, or about ten to 20 proteins, or about ten to 15 proteins, or about 15 to 60 proteins, or about 15 to 55 proteins, or about 15 to 50 proteins, or about 15 to 45 proteins, or about 15 to 40 proteins, or about 15 to 35 proteins, or about 15 to 30 proteins, or about 15 to 25 proteins, or about 15 to 20 proteins, or about 20 to 60 proteins, or about 20 to 55 proteins, or about 20 to 50 proteins, or about 20 to 45 proteins, or about 20 to 40 proteins, or about 20 to 35 proteins, or about 20 to 30 proteins, or about 20 to 25 proteins, or about 25 to 60 proteins, or about 25 to 55 proteins, or about 25 to 50 proteins, or about 25 to 45 proteins, or about 25 to 40 proteins, or about 25 to 30 proteins, or about 30 to 60 proteins, or about 30 to 55 proteins, or about 30 to 50 proteins, or about 30 to 45 proteins, or about 30 to 40 proteins, or about 30 to 35 proteins, or about 35 to 60 proteins, or about 35 to 55 proteins, or about 35 to 50 proteins, or about 35 to 45 proteins, or about 35 to 40 proteins, or about 40 to 60 proteins, or about 40 to 55 proteins, or about 40 to 50 proteins, or about 40 to 45 proteins, or about 45 to 60 proteins, or about 45 to 55 proteins, or about 45 to 50 proteins, or about 50 to 60 proteins, or about 50 to 55 proteins, or about 55 to 60 proteins.

In some embodiments, the kit may also comprise one or more enzymes, substrates, labels, or other components useful for performing the assays.

In some embodiments, the kit further comprises one or more of the following: one or more containers for collecting or holding the sample (e.g., urine sample), controls, directions for performing the methods, any necessary software for analysis and presentation of results.

One skilled in the art will readily recognize that the disclosed one or more components can be readily incorporated into any of the established kit formats that are well known in the art.

### EXAMPLE

Analyses were performed to identify the cervical cancer detection proteins of the present invention.

### Sample Collection

Urine samples were collected from a patient population diagnosed with cervical cancer, and from healthy individuals without cervical cancer.

### Protein Measurement

While any protein measurement technique could have been used, including enzyme-linked immunosorbent assays (ELISA), chemiluminescence immunoassays (CLIA), immunohistochemistry (IHC), liquid-bead immunoassays, mass spectrometry, aptamer-based assays, reverse phase protein arrays (RPPA), etc., a proximity extension assay (PEA) was employed to evaluate proteins in urine. In PEA, each protein was recognized by two antibodies for proper detection. In proximity assays, each of the two antibodies was conjugated to one of two different DNA oligonucleotides, and the reagents were incubated with the samples in solution. The proximity reactions underwent a dilution step. Oligonucleotides on pairs of antibodies that remain in proximity by virtue of having bound the same protein molecule then underwent DNA ligation (proximity ligation assay) or DNA polymerization (proximity extension assay). The effect of the ligation or polymerization reactions was to create amplifiable reporter DNA strands for sensitive readout via, for example, real-time PCR or next-generation sequencing, and the assays could be performed in high multiplex. By constructing the assays so that only proper pairs of antibodies can yield detection signals, but no other combination of antibodies, the detection of many different proteins in parallel was possible without eroding detection specificity by reactions of noncognate pairs.

The analytical performance of the panels was validated for sensitivity, dynamic range, specificity, precision, and scalability. The analytical measuring range was defined by the lower limit of quantification (LLOQ) and upper limit of quantification (ULOQ) and reported in μg/mL. The high dose hook effect (a state of antigen excess relative to the reagent antibodies resulting in falsely lower values) was also determined for each analyte.

All assays were thoroughly validated for precision (repeatability and reproducibility). Intra-assay variation (within-run) was calculated as the mean CV for individual samples, within each separate run during the validation studies. Inter-assay variation (between-runs) was calculated as the mean CV, for the same individual samples, among separate runs during the validation studies. Across the assays, the mean intra-assay and inter-assay variations observed were 8% and 11%, respectively.

Each protein analyte was addressed by a matched pair of antibodies, coupled to unique, partially complementary oligonucleotides and measured by quantitative real-time PCR. Validation of the readout specificity for all of the panels was carried out using a simple, sequential approach in which pools of protein analytes were tested.

### Feature Selection

Proteins were used to create features that could be used for the classification of samples. The proteins were categorized based on their concentration or their patterns of change detected by different statistical or machine-learning techniques to create new features.

Machine learning and statistical analyses techniques used to generate features and the final score for the cancer were included but not limited to the following concepts and methods: supervised learning concepts that may include AODE; artificial neural network, such as Backpropagation, Auto encoders, Hopfield networks, Boltzmann machines, Restricted Boltzmann Machines, and Spiking neural networks; Bayesian statistics, such as Bayesian network and Bayesian knowledge base; case-based reasoning; Gaussian process regression; gene expression programming: group method of data handling (GMDH); inductive logic programming; instance-based learning; lazy learning; learning Automata; learning vector quantization: logistic model tree; minimum message length (decision trees, decision graphs, etc.), such as nearest neighbor algorithm and analogical modeling; probability approximately correct learning (PAC) learning; ripple down rules, a knowledge acquisition methodology; symbolic machine learning algorithms; support vector machines; random forests; ensembles of classifiers, such as bootstrap aggregating (bagging) and boosting (meta-algorithm); ordinal classification: information fuzzy networks (IFN); conditional random field; ANOVA; linear classifiers, such’as Fisher's linear discriminant, linear regression, logistic regression, multinomial logistic relion, naive Bayes classifier, Perceptron, support vector machines; quadratic classifiers; k-nearest neighbor; boosting; decision trees, such as C4.5, random forests, ID3, CART, SLIQ SPRINT; Bayesian netI, such as Naive Bayes; and Hidden Markov models. cUnsupervised learning concepts may include; expectation-maximization algorithm; vector quantization; generative topographic map; information bottleneck method; artificial neural network, such as self-organizing map: association rule learning, such as Apriori algorithm. Eclat algorithm, and FP growth algorithm; hierarchical clusterings such as single linkage clustering and conceptual clustering; cluster analysis, such as K-means algorithm, fuzzy clustering, DBSCAN, and OPTICS algorithm; and outlier detection, such as local outlier factor. Semi-supervised learning concepts may include: generative models; low-density separation: graph-based methods, and co-training. Reinforcement learning concepts may include temporal difference learning; Q-learning, learning automata, and SARSA. Deep learning concepts may include deep belief networks; deep Boltzmann machines; deep convolutional neural networks; deep recurrent neural networks; and hierarchical temporal memory.

### Cervical Cancer Detection Proteins

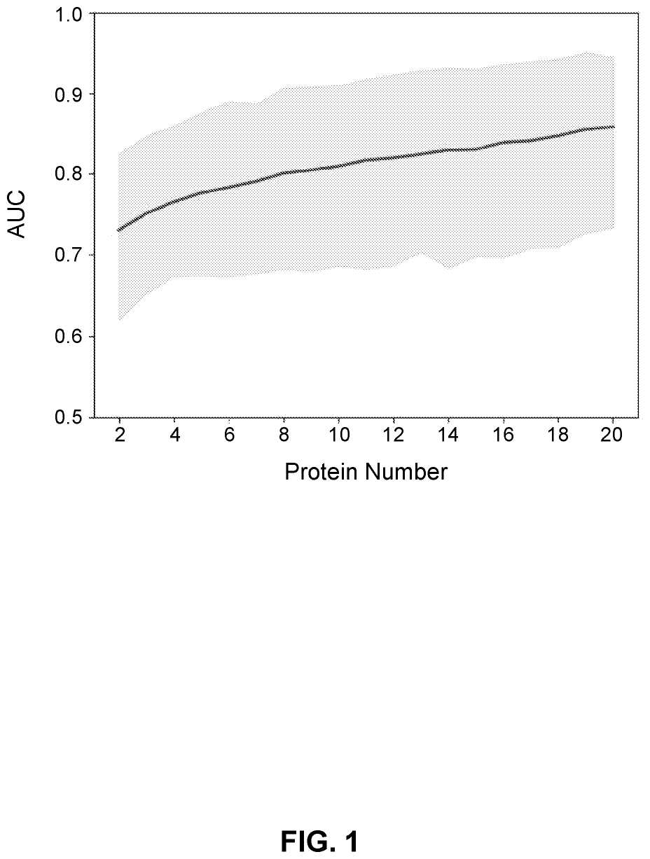

One or more features were fed into one or more computation models. The classifiers were used to calculate a score for the patient. The scores of different classifiers were combined to identify the patient as having cervical cancer or not. The computational model only selected protein or protein combinations that could generate a receiver operating characteristic (ROC) curve of greater than or equal to 0.6. The resulting cervical cancer detection proteins are shown in Table 1. FIG. 1 shows that the accuracy is over 0.7 when any two proteins through any 20 proteins are randomly selected.

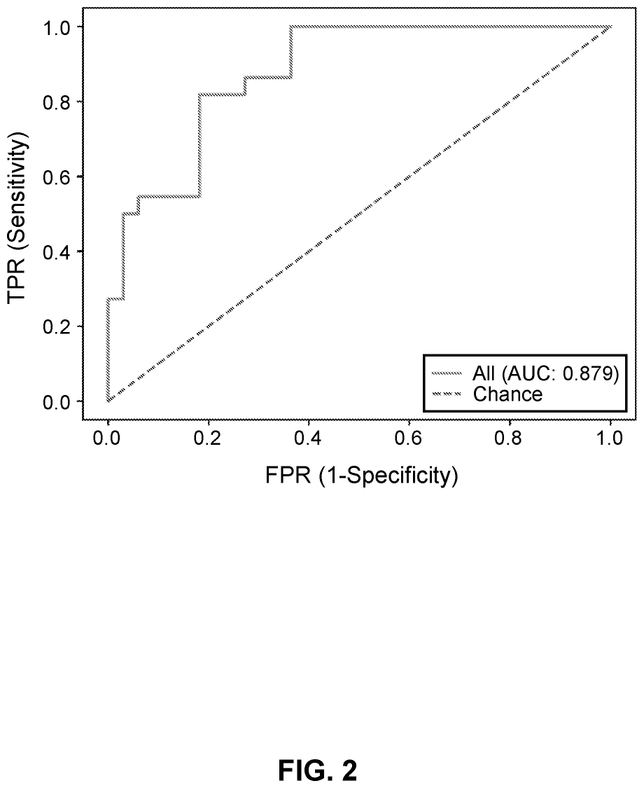

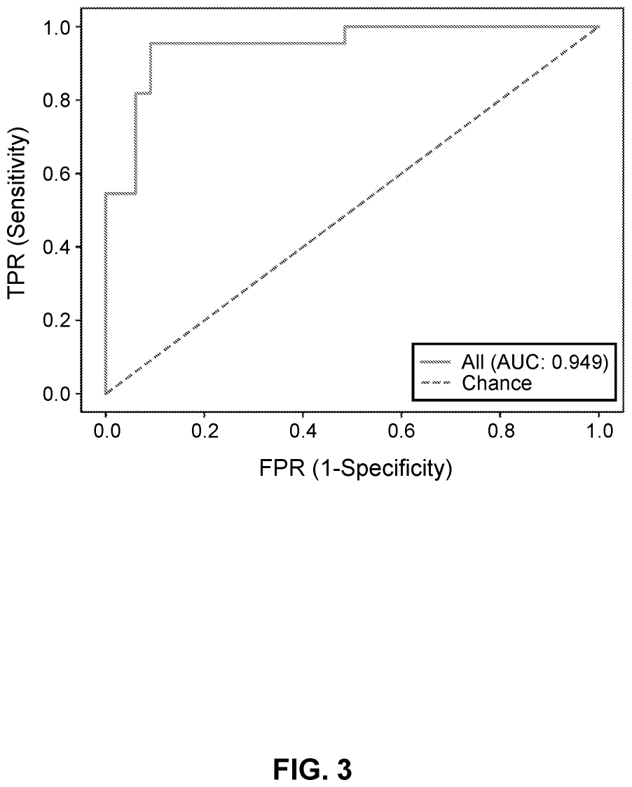

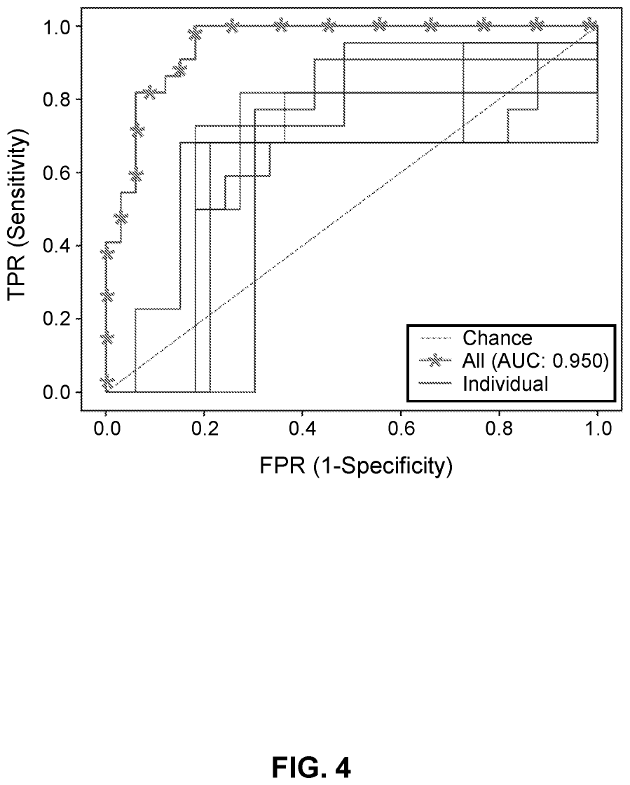

The model also identified particular substes of the proteins of Table 1 from which one or more proteins can be selected from to detect cervical cancer. Such subsets are presented in Table 2, Table 3, and Table 4. In addition, it was determined that panels of the proteins of Table 2, Table 3, and Table 4 each exhibits high diagnostic utility: the ROC curve generated from the panel of all of the proteins listed in Table 2 has an AUC of about 0.879 (see FIG. 2), the ROC curve generated from the panel of all of the proteins listed in Table 3 has an AUC of about 0.949 (see FIG. 3), and the ROC curve generated from the panel of all of the proteins listed in Table 4 has an AUC of about 0.950 (see FIG. 4).

TABLE 1
Cervical cancer detection proteins.

| Gene Name | Uniprot ID | AUC | Gene Name | Uniprot ID | AUC |
|---|---|---|---|---|---|
| NDUFB7 | P17568 | 0.813 | C9 | P02748 | 0.729 |
| S100A4 | P26447 | 0.700 | FAM13A | O94988 | 0.716 |
| SLC27A4 | Q6P1M0 | 0.770 | GYS1 | P13807 | 0.657 |
| SCIN | Q9Y6U3 | 0.740 | BLVRB | P30043 | 0.697 |
| DGCR6 | Q14129 | 0.685 | TPD52L2 | O43399 | 0.736 |
| TIMM10 | P62072 | 0.674 | ASPSCR1 | Q9BZE9 | 0.650 |
| NAMPT | P43490 | 0.734 | PRDX6 | P30041 | 0.715 |
| LAMB1 | P07942 | 0.713 | GSTP1 | P09211 | 0.734 |
| FDX1 | P10109 | 0.712 | MAD1L1 | Q9Y6D9 | 0.734 |
| SLITRK1 | Q96PX8 | 0.690 | PMS1 | P54277 | 0.642 |
| CGB3; | P0DN86 | 0.753 | BAP18 | Q8IXM2 | 0.709 |
| CGB5; | | | | | |
| CGB8 | | | | | |
| LELP1 | Q5T871 | 0.618 | HIP1R | O75146 | 0.719 |
| AHSG | P02765 | 0.720 | CORO1A | P31146 | 0.595 |
| MAPK13 | O15264 | 0.702 | TTR | P02766 | 0.579 |
| CDA | P32320 | 0.741 | ADAMTS1 | Q9UHI8 | 0.634 |
| PPP3R1 | P63098 | 0.775 | FLT3 | P36888 | 0.680 |
| MCEMP1 | Q8IX19 | 0.645 | TAX1BP1 | Q86VP1 | 0.704 |
| CLU | P10909 | 0.738 | ARFIP1 | P53367 | 0.638 |
| FSHB | P01225 | 0.741 | ACE | P12821 | 0.617 |
| FGF16 | O43320 | 0.736 | MENT | Q9BUN1 | 0.617 |
| RNASE10 | Q5GAN6 | 0.652 | PGR | P06401 | 0.620 |
| ATXN3 | P54252 | 0.771 | CYB5A | P00167 | 0.676 |
| S100A12 | P80511 | 0.697 | INPPL1 | O15357 | 0.672 |
| CHCHD6 | Q9BRQ6 | 0.791 | NXPE4 | Q6UWF7 | 0.669 |
| RLN2 | P04090 | 0.665 | MFAP5 | Q13361 | 0.620 |
| NAGA | P17050 | 0.744 | NDUFS6 | O75380 | 0.736 |
| RNASE3 | P12724 | 0.676 | PIBF1 | Q8WXW3-4 | 0.653 |
| AKR7L | Q8NHP1 | 0.725 | PCBP2 | Q15366 | 0.645 |
| COMT | P21964 | 0.720 | PGM2 | Q96G03 | 0.709 |
| RRAS | P10301 | 0.567 | DENND2B | P78524 | 0.671 |

TABLE 2
Subset of cervical cancer detection proteins from Table
1, which together can achieve an AUC of 0.879.

| Gene Name | Uniprot ID | Gene Name | Uniprot ID |
|---|---|---|---|
| NDUFB7 | P17568 | CLU | P10909 |
| CHCHD6 | Q9BRQ6 | NDUFS6 | O75380 |
| PPP3R1 | P63098 | FGF16 | O43320 |
| ATXN3 | P54252 | TPD52L2 | O43399 |
| SLC27A4 | Q6P1M0 | GSTP1 | P09211 |
| CGB3; | P0DN86 | NAMPT | P43490 |
| CGB5; | | | |
| CGB8 | | | |
| NAGA | P17050 | MAD1L1 | Q9Y6D9 |
| FSHB | P01225 | C9 | P02748 |
| CDA | P32320 | AKR7L | Q8NHP1 |
| SCIN | Q9Y6U3 | AHSG | P02765 |

TABLE 3
Subset of cervical cancer detection proteins from Table
1, which together can achieve an AUC of 0.949.

| Gene Name | Uniprot ID | Gene Name | Uniprot ID |
|---|---|---|---|
| NDUFB7 | P17568 | TPD52L2 | O43399 |
| S100A4 | P26447 | TTR | P02766 |
| SLC27A4 | Q6P1M0 | ADAMTS1 | Q9UHI8 |
| SCIN | Q9Y6U3 | FLT3 | P36888 |
| DGCR6 | Q14129 | TAX1BP1 | Q86VP1 |
| TIMM10 | P62072 | ARFIP1 | P53367 |
| NAMPT | P43490 | ACE | P12821 |
| LAMB1 | P07942 | MENT | Q9BUN1 |
| CGB3; | P0DN86 | PGR | P06401 |
| CGB5; | | | |
| CGB8 | | | |
| MAPK13 | O15264 | CYB5A | P00167 |
| CDA | P32320 | INPPL1 | O15357 |
| PPP3R1 | P63098 | NXPE4 | Q6UWF7 |
| CLU | P10909 | MFAP5 | Q13361 |
| FSHB | P01225 | NDUFS6 | O75380 |
| FGF16 | O43320 | PIBF1 | Q8WXW3-4 |
| RNASE10 | Q5GAN6 | PCBP2 | Q15366 |
| ATXN3 | P54252 | PGM2 | Q96G03 |
| CHCHD6 | Q9BRQ6 | DENND2B | P78524 |
| GYS1 | P13807 | | |

TABLE 4
Subset of cervical cancer detection proteins from Table
1, which together can achieve an AUC of 0.950.

| Gene Name | Uniprot ID | Gene Name | Uniprot ID |
|---|---|---|---|
| NDUFB7 | P17568 | RNASE10 | Q5GAN6 |
| FSHB | P01225 | TIMM10 | P62072 |
| DGCR6 | Q14129 | SLC27A4 | Q6P1M0 |
| MAPK13 | O15264 | | |

### EMBODIMENTS

Select embodiments of the present invention are as follows:

Embodiment 1. A method of evaluating a subject for cervical cancer, the method comprising:
determining in a biological sample from the subject a concentration of one or more proteins selected from Table 1;
thereby evaluating the subject for cancer.
   Embodiment 2. The method of Embodiment 1, further comprising applying a classifier to the concentration of the one or more proteins that identifies whether the concentration of the one or more proteins is indicative of the subject having cervical cancer.
   Embodiment 3. The method of Embodiment 1 or 2, further comprising administering a treatment to the subject.
   Embodiment 4. A method of treating cervical cancer in a subject, comprising
(a) acquiring results from the method of Embodiments 1 or 2; and
(b) administering a treatment to the subject.
   Embodiment 5. The method of Embodiment 4, wherein the treatment is responsive to the results acquired in (a).
   Embodiment 6. The method of Embodiment 4 or 5, wherein (a) comprises:
(i) determining in a biological sample from the subject a concentration of one or more proteins selected from Table 1; and
(ii) applying a classifier to the concentration of the one or more proteins to identify whether the subject has cervical cancer.
   Embodiment 7. A method of treating cervical cancer in a subject, the method comprising:
(a) acquiring results from an evaluation of the subject that determined the subject has cervical cancer;
(b) administering a treatment to the subject,
wherein the evaluation comprises:
   (i) determining in a biological sample from the subject a concentration of one or more proteins selected from Table 1; and
   (ii) applying a classifier to the concentration of the one or more proteins to identify whether the subject has cervical cancer.
      Embodiment 8. The method of any one of Embodiments 4-8, wherein the results in (a) are acquired from a third party.
      Embodiment 9. A method of detecting cervical cancer in a subject, the method comprising:
determining in a biological sample from the subject a concentration of one or more proteins selected from Table 1; and
applying a classifier to the concentration of the one or more proteins that identifies whether the concentration of the one or more proteins is indicative that cervical cancer is detected.
   Embodiment 10. The method of Embodiment 9, further comprising administering a treatment to the subject.
   Embodiment 11. A method of treating cervical cancer in a subject, comprising
(a) acquiring results from the method of Embodiment 9; and
(b) administering a treatment to the subject.
   Embodiment 12. The method of Embodiment 11, wherein the treatment is responsive to the results acquired in (a).
   Embodiment 13. A method of treating cervical cancer in a subject, the method comprising
determining in a biological sample from the subject a concentration of one or more proteins selected from Table 1;
applying a classifier to the concentration of the one or more proteins that identifies whether the concentration of the one or more proteins is indicative that cervical cancer is detected; and
administering a treatment to the subject when cervical cancer is detected.
   Embodiment 14. A method of treating cervical cancer in a subject in whom cervical cancer was detected, the method comprising administering a treatment for cervical cancer to the subject, wherein cervical cancer was detected in the subject by a method comprising:
determining in a biological sample from the subject a concentration of one or more proteins selected from Table 1; and
applying a classifier to the concentration of the one or more proteins that identifies whether the concentration of the one or more proteins is indicative that cervical cancer is detected.
   Embodiment 15. The method of Embodiment 14, wherein the method of detecting cervical cancer was performed by a third party.
   Embodiment 16. The method of any one of Embodiments 1-15, wherein the cervical cancer is early-stage.
   Embodiment 17. The method of any one of Embodiments 1-16, wherein the subject is asymptomatic of cervical cancer.
   Embodiment 18. The method of Embodiment 17, wherein the subject is undergoing a screen for cervical cancer.
   Embodiment 19. The method of any one of Embodiments 1-18, wherein the subject is symptomatic of cervical cancer.
   Embodiment 20. A method of evaluating a treatment for cervical cancer in a subject, the method comprising:
administering a treatment for cervical cancer, and
determining in a biological sample from the subject a concentration of one or more proteins selected from Table 1;
thereby evaluating the treatment.
   Embodiment 21. A method of evaluating the efficacy of a treatment for cervical cancer in a subject, the method comprising
(a) administering a treatment for cervical cancer to the subject, and
(b) determining in a biological sample from the subject a concentration of one or more proteins selected from Table 1;
thereby evaluating the efficacy of the treatment.
   Embodiment 22. A method of treating cervical cancer in a subject, the method comprising
(a) administering a treatment for cervical cancer to the subject, and
(b) determining in a biological sample from the subject a concentration of one or more proteins to evaluate the efficacy of the treatment, wherein the one or more proteins are selected from Table 1.
   Embodiment 23. A method of adjusting a treatment for cervical cancer in a subject, the method comprising
(a) administering a treatment for cervical cancer to the subject,
(b) determining in a biological sample from the subject a concentration of one or more proteins, wherein the one or more proteins are selected Table 1, and
(c) administering an adjusted treatment to the subject when it is determined that the adjusted treatment is necessary.
   Embodiment 24. A method of treating cervical cancer in a subject, the method comprising
(a) administering a treatment for cervical cancer to the subject, and
(b) determining in a biological sample from the subject a concentration of one or more proteins to evaluate whether the treatment requires adjustment, wherein the one or more proteins are selected from Table 1.
   Embodiment 25. The method of Embodiment 24, further comprising administering an adjusted treatment when it is determined that the adjusted treatment is necessary.
   Embodiment 26. A method of monitoring for cervical cancer recurrence in a subject, comprising
(a) administering a treatment for cervical cancer to the subject, and
(b) determining in a biological sample from the subject a concentration of one or more proteins to evaluate whether the cervical cancer is recurring, wherein the one or more proteins are selected from Table 1.
   Embodiment 27. A method of treating cervical cancer in a subject, the method comprising
(a) administering a treatment for cervical cancer to the subject, and
(b) determining in a biological sample from the subject a concentration of one or more proteins to evaluate whether cancer is recurring, wherein the one or more proteins are selected from Table 1.
   Embodiment 28. The method of Embodiment 26 or 27, further comprising administering a second treatment when it is determined that the cancer is recurring.
   Embodiment 29. The method of any one of Embodiments 20-28, further comprising applying a classifier to the concentration of the one or more proteins that identifies whether the concentration of the one or more proteins is indicative of the subject having cervical cancer.
   Embodiment 30. The method of any one of Embodiments 1-29, wherein the biological sample is selected from a plasma sample, serum sample, saliva sample, CSF sample, sweat sample, urine sample, or tear sample.
   Embodiment 31. The method of Embodiment 30, wherein the biological sample is a urine sample.
   Embodiment 32. The method of any one of Embodiments 1-31, further comprising collecting the biological sample from the subject.
   Embodiment 33. The method of Embodiment 32, wherein the collection of the biological sample is performed in the home of the subject.
   Embodiment 34. The method of Embodiment 33, wherein the collection of the biological sample is performed in a medical facility.
   Embodiment 35. The method of any one of Embodiments 1-34, wherein the determination of the concentration of the one or more proteins is performed in the home of the subject.
   Embodiment 36. The method of any one of Embodiments 1-34, wherein the determination of the concentration of the one or more proteins is performed in a medical facility.
   Embodiment 37. The method of any one of Embodiments 1-36, wherein the number of proteins for which the concentration is determined is sufficient to achieve an area-under-the-curve (AUC) of a ROC curve of at least about 0.6.
   Embodiment 38. The method of Embodiment 37, wherein the number of proteins for which the concentration is determined is sufficient to achieve an AUC of a ROC curve of at least about 0.7.
   Embodiment 39. The method of Embodiment 38, wherein the number of proteins for which the concentration is determined is sufficient to achieve an AUC of a ROC curve of at least about 0.8.
   Embodiment 40. The method of any one of Embodiments 1-39, wherein the concentration of the two or more proteins is determined by one or more assays.
   Embodiment 41. The method of any one of Embodiments 20-40, wherein the administration of the treatment in (a) is performed by a third party.
   Embodiment 42. The method of any one of Embodiments 20-40, wherein the determination in a urine sample from the subject a concentration of one or more proteins in (b) is performed by a third party.
   Embodiment 43. A method of measuring amounts of proteins in a subject, the method comprising determining individual amounts of one or more proteins selected from Table 1.
   Embodiment 44. The method of any one of Embodiments 1-43, wherein the one or more proteins are selected from Table 2.
   Embodiment 45. The method of any one of Embodiments 1-43, wherein the one or more proteins are selected from Table 3.
   Embodiment 46. The method of any one of Embodiments 1-43, wherein the one or more proteins are selected from Table 4.
   Embodiment 47. The method of any one of Embodiments 1-46, wherein two or more proteins are selected.
   Embodiment 48. The method of any one of Embodiments 1-46, wherein three or more proteins are selected.
   Embodiment 49. The method of any one of Embodiments 1-46, wherein five or more proteins are selected.
   Embodiment 50. The method of any one of Embodiments 1-45, wherein ten or more proteins are selected.
   Embodiment 51. The method of any one of Embodiments 1-43 or 45, wherein 20 or more proteins are selected.
   Embodiment 52. The method of any one of Embodiments 1-43 or 45, wherein 30 or more proteins are selected.
   Embodiment 53. The method of any one of Embodiments 1-43, wherein 40 or more proteins are selected.
   Embodiment 54. The method of any one of Embodiments 1-43, wherein 50 or more proteins are selected.
   Embodiment 55. The method of any one of Embodiments 1-46, wherein all proteins are selected.
   Embodiment 56. The method of any one of Embodiments 1-43, wherein no more than about 50 proteins are selected.
   Embodiment 57. The method of any one of Embodiments 1-43, wherein no more than about 40 proteins are selected.
   Embodiment 58. The method of any one of Embodiments 1-43 or 45, wherein no more than about 30 proteins are selected.
   Embodiment 59. The method of any one of Embodiments 1-45, wherein no more than about 20 proteins are selected.
   Embodiment 60. The method of any one of Embodiments 1-45, wherein no more than about ten proteins are selected.
   Embodiment 61. The method of any one of Embodiments 1-46, wherein no more than about five proteins are selected.

### REFERENCES

Arbyn M, et al., Estimates of incidence and mortality of cervical cancer in 2018: a worldwide analysis, Lancet Glob. Health. 2022, 10: e41.
Jing J., Urine biomarkers in the early stages of diseases: current status and perspective. Discov. Med., 2018, 25: 57-65.
Siegel R L, et al., Cancer statistics. 2023, CA Cancer J Clin., 2022, 72: 7-33.
Wright J D, et al., Prognostic performance of the 2018 International Federation of Gynecology and Obstetrics cervical cancer staging guidelines, Obstet. Gynecol., 2019, 134: 49-57.

1. A method of evaluating a subject for cervical cancer, the method comprising:
determining in a biological sample from the subject a concentration of one or more proteins selected from Table 1;
thereby evaluating the subject for cancer.

2. The method of claim 1, further comprising applying a classifier to the concentration of the one or more proteins that identifies whether the concentration of the one or more proteins is indicative of the subject having cervical cancer.

3. The method of claim 1 or 2, further comprising administering a treatment to the subject.

4. A method of treating cervical cancer in a subject, comprising
(a) acquiring results from the method of claim 1 or 2; and
(b) administering a treatment to the subject.

5. The method of claim 4, wherein the treatment is responsive to the results acquired in (a).

6. The method of claim 4 or 5, wherein (a) comprises:
(i) determining in a biological sample from the subject a concentration of one or more proteins selected from Table 1; and
(ii) applying a classifier to the concentration of the one or more proteins to identify whether the subject has cervical cancer.

7. A method of treating cervical cancer in a subject, the method comprising:
(a) acquiring results from an evaluation of the subject that determined the subject has cervical cancer;
(b) administering a treatment to the subject,
wherein the evaluation comprises:
(i) determining in a biological sample from the subject a concentration of one or more proteins selected from Table 1; and
(ii) applying a classifier to the concentration of the one or more proteins to identify whether the subject has cervical cancer.

8. The method of any one of claims 4-8, wherein the results in (a) are acquired from a third party.

9. A method of detecting cervical cancer in a subject, the method comprising:
determining in a biological sample from the subject a concentration of one or more proteins selected from Table 1; and
applying a classifier to the concentration of the one or more proteins that identifies whether the concentration of the one or more proteins is indicative that cervical cancer is detected.

10. The method of claim 9, further comprising administering a treatment to the subject.

11. A method of treating cervical cancer in a subject, comprising
(a) acquiring results from the method of claim 9; and
(b) administering a treatment to the subject.

12. The method of claim 11, wherein the treatment is responsive to the results acquired in (a).

13. A method of treating cervical cancer in a subject, the method comprising
determining in a biological sample from the subject a concentration of one or more proteins selected from Table 1;
applying a classifier to the concentration of the one or more proteins that identifies whether the concentration of the one or more proteins is indicative that cervical cancer is detected; and
administering a treatment to the subject when cervical cancer is detected.

14. A method of treating cervical cancer in a subject in whom cervical cancer was detected, the method comprising administering a treatment for cervical cancer to the subject, wherein cervical cancer was detected in the subject by a method comprising:
determining in a biological sample from the subject a concentration of one or more proteins selected from Table 1; and
applying a classifier to the concentration of the one or more proteins that identifies whether the concentration of the one or more proteins is indicative that cervical cancer is detected.

15. The method of claim 14, wherein the method of detecting cervical cancer was performed by a third party.

16. The method of any one of claims 1-15, wherein the cervical cancer is early-stage.

17. The method of any one of claims 1-16, wherein the subject is asymptomatic of cervical cancer.

18. The method of claim 17, wherein the subject is undergoing a screen for cervical cancer.

19. The method of any one of claims 1-18, wherein the subject is symptomatic of cervical cancer.

20. A method of evaluating a treatment for cervical cancer in a subject, the method comprising:
(a) administering a treatment for cervical cancer, and
(b) determining in a biological sample from the subject a concentration of one or more proteins selected from Table 1;
thereby evaluating the treatment.

21. A method of evaluating the efficacy of a treatment for cervical cancer in a subject, the method comprising
(a) administering a treatment for cervical cancer to the subject, and
(b) determining in a biological sample from the subject a concentration of one or more proteins selected from Table 1;
thereby evaluating the efficacy of the treatment.

22. A method of treating cervical cancer in a subject, the method comprising
(a) administering a treatment for cervical cancer to the subject, and
(b) determining in a biological sample from the subject a concentration of one or more proteins to evaluate the efficacy of the treatment, wherein the one or more proteins are selected from Table 1.

23. A method of adjusting a treatment for cervical cancer in a subject, the method comprising
(a) administering a treatment for cervical cancer to the subject,
(b) determining in a biological sample from the subject a concentration of one or more proteins, wherein the one or more proteins are selected Table 1, and
(c) administering an adjusted treatment to the subject when it is determined that the adjusted treatment is necessary.

24. A method of treating cervical cancer in a subject, the method comprising
(a) administering a treatment for cervical cancer to the subject, and
(b) determining in a biological sample from the subject a concentration of one or more proteins to evaluate whether the treatment requires adjustment, wherein the one or more proteins are selected from Table 1.

25. The method of claim 24, further comprising administering an adjusted treatment when it is determined that the adjusted treatment is necessary.

26. A method of monitoring for cervical cancer recurrence in a subject, the method comprising
(a) administering a treatment for cervical cancer to the subject, and
(b) determining in a biological sample from the subject a concentration of one or more proteins to evaluate whether the cervical cancer is recurring, wherein the one or more proteins are selected from Table 1.

27. A method of treating cervical cancer in a subject, the method comprising
(a) administering a treatment for cervical cancer to the subject, and
(b) determining in a biological sample from the subject a concentration of one or more proteins to evaluate whether cancer is recurring, wherein the one or more proteins are selected from Table 1.

28. The method of claim 26 or 27, further comprising administering a second treatment when it is determined that the cancer is recurring.

29. The method of any one of claims claim 20-28, further comprising applying a classifier to the concentration of the one or more proteins that identifies whether the concentration of the one or more proteins is indicative of the subject having cervical cancer.

30. The method of any one of claims 1-29, wherein the biological sample is selected from a plasma sample, serum sample, saliva sample, CSF sample, sweat sample, urine sample, or tear sample.

31. The method of claim 30, wherein the biological sample is a urine sample.

32. The method of any one of claims 1-31, further comprising collecting the biological sample from the subject.

33. The method of claim 32, wherein the collection of the biological sample is performed in the home of the subject.

34. The method of claim 33, wherein the collection of the biological sample is performed in a medical facility.

35. The method of any one of claims 1-34, wherein the determination of the concentration of the one or more proteins is performed in the home of the subject.

36. The method of any one of claims 1-34, wherein the determination of the concentration of the one or more proteins is performed in a medical facility.

37. The method of any one of claims 1-36, wherein the number of proteins for which the concentration is determined is sufficient to achieve an area-under-the-curve (AUC) of a ROC curve of at least about 0.6.

38. The method of claim 37, wherein the number of proteins for which the concentration is determined is sufficient to achieve an AUC of a ROC curve of at least about 0.7.

39. The method of claim 38, wherein the number of proteins for which the concentration is determined is sufficient to achieve an AUC of a ROC curve of at least about 0.8.

40. The method of any one of claims 1-39, wherein the concentration of the two or more proteins is determined by one or more assays.

41. The method of any one of claims 20-40, wherein the administration of the treatment in (a) is performed by a third party.

42. The method of any one of claims 20-40, wherein the determination in a urine sample from the subject a concentration of one or more proteins in (b) is performed by a third party.

43. A method of measuring amounts of proteins in a subject, the method comprising determining individual amounts of one or more proteins selected from Table 1.

44. The method of any one of claims 1-43, wherein the one or more proteins are selected from Table 2.

45. The method of any one of claims 1-43, wherein the one or more proteins are selected from Table 3.

46. The method of any one of claims 1-43, wherein the one or more proteins are selected from Table 4.

47. The method of any one of claims 1-46, wherein two or more proteins are selected.

48. The method of any one of claims 1-46, wherein three or more proteins are selected.

49. The method of any one of claims 1-46, wherein five or more proteins are selected.

50. The method of any one of claims 1-45, wherein ten or more proteins are selected.

51. The method of any one of claims 1-43 or 45, wherein 20 or more proteins are selected.

52. The method of any one of claims 1-43 or 45, wherein 30 or more proteins are selected.

53. The method of any one of claims 1-43, wherein 40 or more proteins are selected.

54. The method of any one of claims 1-43, wherein 50 or more proteins are selected.

55. The method of any one of claims 1-46, wherein all proteins are selected.

56. The method of any one of claims 1-43, wherein no more than about 50 proteins are selected.

57. The method of any one of claims 1-43, wherein no more than about 40 proteins are selected.

58. The method of any one of claims 1-43 or 45, wherein no more than about 30 proteins are selected.

59. The method of any one of claims 1-43 or 45, wherein no more than about 20 proteins are selected.

60. The method of any one of claims 1-45, wherein no more than about ten proteins are selected.

61. The method of any one of claims 1-46, wherein no more than about five proteins are selected.
